(19) United States
(12) Patent Application Publication
(10) Pub. No.: US 2019/0115111 A1
(43) Pub. Date: Apr. 18, 2019

(54) SYSTEM AND METHOD FOR SHARING MEDICAL TEST RESULTS

(71) Applicant: Roody Mesidor, Rosedale, MD (US)

(72) Inventor: Roody Mesidor, Rosedale, MD (US)

(21) Appl. No.: 16/162,733

(22) Filed: Oct. 17, 2018

Related U.S. Application Data
(60) Provisional application No. 62/573,653, filed on Oct. 17, 2017.

(51) Int. Cl. G16H 80/00; G16H 15/00; G16H 10/60

(52) U.S. Cl. CPC G16H 80/00; G16H 10/60; G16H 15/00

(57) ABSTRACT

Described is a computer-implemented method and system for physicians, patients, and third parties to share medical-test result and other health-related information. This method and system can utilize a web portal, desktop computer software, and/or mobile app.

### CROSS REFERENCE TO RELATED APPLICATIONS

This Application claims the benefit of U.S. Provisional Application Ser. No. 62/573,653 filed Oct. 17, 2017, the disclosure of which is incorporated by reference herein.

### COPYRIGHT NOTICE

This patent document contains material that is subject to copyright protection. The copyright owner has no objection to the reproduction of this patent document or related materials as they appear in the files of the Patent Office of the United States or other countries, but otherwise reserves all other intellectual property rights including copyrights.

### BACKGROUND

Despite many efforts to digitize the medical records of patients, most patients must call their doctor's office, schedule a follow-up visit, or log into a specialized portal in order to receive test results. The process is time consuming, and less than ideal. Many times, patients can only receive a copy of their medical records in paper form. This means that if a patient would like to share parts of their medical records with a third party, then the patient generally must send that third party a paper copy of their records. Such records may contain sensitive information about the patient that may be beyond the scope of what that patient would like to share.

### SUMMARY

Described is a computer-implemented method and system (such as a mobile application) for physicians, patients, and third parties to share medical test data and other health-related information.

In one aspect, the system and method allow a physician to share medical-test result with a patient directly to the patient's device. For instance, the physician can either log into an online portal, use an app on the physician's device, or use a program to upload the patient's medical-test result to a server. Likewise, the patient will download an app on their device or visit an online portal that then gives them access to the medical-test result uploaded by the physician. Thereby, the physician can forward test results or other health information directly to the patient via an app on the patient's device or through a web portal.

In another embodiment, the physician connects directly to the app on the patient's device and uploads the medical-test result directly onto the patient's device. This can be done through several means such as connecting over wi-fi or near-field communication. If the physician provides the patient with a paper copy of the test result, then the patient can upload the test result into the app by either scanning or taking a picture of the test result. In this embodiment, the patient can send the test result to the server directly from their device over an internet connection. Once sent to the server, a medical data specialist or the server itself can verify the authenticity of the test result by looking for evidence of tampering and comparing the test result against previously authenticated examples.

In another aspect, the system and method allows a user (such as the patient) to share their medical-test results with third parties. For instance, a user can create a profile online either through the app or an online portal. After the profile is created, the user is assigned a unique identifier (such as unique number). The user may then share the unique identifier with his/her physician or third parties. Based on the unique identifier, a physician may upload test results associated with that unique identifier. After the results are uploaded, a third party—authorized by the user—may view the test results, which are associated with a unique identifier. The third party can then view the user's medical-test results. In another embodiment, the third party is not able to view the medical-test results themselves, but instead the third party will be shown a checkmark indicating whether or not each of the user's medical-test results reflected a positive health outcome. The checkmark may have a variety of colors indicating how long ago the user received each medical test. For instance, the checkmark may be green if the medical test was performed less than three months ago, yellow if less than six months ago, and red if greater than six months ago.

In another aspect, each test result will have a unique identifier assigned to it. If a third party desires to view a test result, the third party can use the unique identifier associated with the test result, provided by the user (patient) of the app, to view the test result.

In another aspect, physicians and users/patients can communicate via the app, and users can subscribe to a service via the app to obtain or share test results.

The app will make it easier for people entering into relationships to know their partner's medical status, such as whether the other person has recently been tested for a sexually-transmitted disease (STD). Because the test results may not be manipulated, and arrive directly from a verified physician, people may rely on the medical-test result as being “authentic.” In other words, the app helps to foster authenticity, because results cannot be counterfeited or faked.

In addition, the app will make it easier for family members to know each other's medical statuses; especially for the elderly family members. Patients won't have to keep calling a physician's office (often waiting on hold) for exam or test results. Additionally, patients can opt out of a follow-up visit after receiving test results through the system if the results reflect a positive health outcome.

In yet another aspect, the app will request some form of verified identification (federal or state) to verify the profile. And every profile will have a unique identifier linked to the identification.

With respect to medical-test result, including STD test results, the app may also provide recent trend analysis in the user's area, to warn and educate the user.

The user may also choose limited or unlimited sharing of medical-test result via the app.

For example, in a limited-sharing mode, the app may allow a person to view a test with only limited individually identifiable information such as (white female—age 21), the profile's unique identifier, and test result. To further protect the user's identity, when the app is in a limited-sharing mode, after a test result is viewed by a third party, the test result will automatically be deleted from the app after viewing, such as one hour after viewing.

In an unlimited-sharing mode, the user may view more personally identifiable information associated with the medical-test result, such as the unique identifier, name, date of birth, age, and profile of the user. In the unlimited-sharing mode, the test results will remain indefinitely unless removed from the server or user's device.

In terms of authenticity, only a certified physician (or other certified/verified health-care provider) can upload results to the server or app. And only the user/patient can—at least initially—view their results. Thereafter, the user/patient may choose to share it with others in a limited or unlimited manner.

The system and method may be implemented as a mobile application, web service, and/or computer software application. In one aspect the system and method stores attributes associated with each user in a database, constructs a health model about the user, or some other aspect of the users. The model may be used to report and/or predict intelligent information that benefits the patient or healthcare provider.

This description is provided to introduce a selection of concepts in a simplified form. This description is not necessarily intended to identify key features or essential features of the claimed subject matter, nor is it necessarily intended to be used as an aid in determining the scope of the claimed subject matter.

This summary is provided to introduce a selection of concepts in a simplified form that are further described below. This summary is not necessarily intended to identify key features or essential features of the claimed subject matter, nor is it necessarily intended to be used as an aid in determining the scope of the claimed subject matter.

The foregoing outlines examples of this disclosure so that those skilled in the relevant art may better understand the detailed description that follows. Additional embodiments and details will be described hereinafter. Those skilled in the relevant art should appreciate that they can readily use any of these disclosed embodiments as a basis for designing or modifying other structures or functions for carrying out the invention, without departing from the spirit and scope of the invention.

Reference herein to “one embodiment,” “an embodiment,” “an aspect,” “an implementation,” “an example,” or similar formulations, means that a particular feature, structure, operation, or characteristic described in connection with the embodiment is included in at least one embodiment of the present invention. Thus, different appearances of such phrases or formulations herein do not necessarily refer to the same embodiment. Furthermore, various particular features, structures, operations, or characteristics may be combined in any suitable manner in one or more embodiments.

### BRIEF DESCRIPTION OF THE DRAWINGS

The detailed description is described with reference to the accompanying figures. In the figures, the left-most digit(s) of a reference number identifies the figure in which the reference number first appears. The figures are not necessarily drawn to scale.

FIG. 1 shows an embodiment of the system and method utilizing an electronic data server and medical data specialist.

FIG. 2 shows an embodiment of the system and method utilizing an electronic data server, but not a medical data specialist.

FIG. 3 shows an embodiment of the system and method where users upload medical-test results to an electronic data server.

FIG. 4 shows an embodiment of the system and method that does not utilize an electronic data server nor a medical data specialist.

FIG. 5 shows a diagram of an embodiment of the system and method.

### DETAILED DESCRIPTION

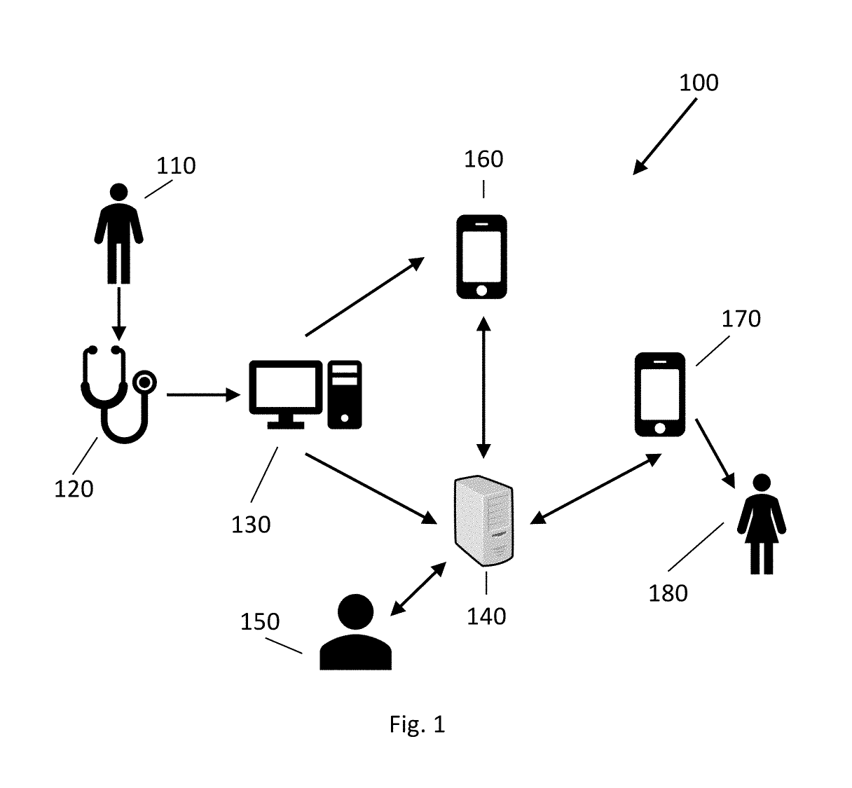

Described is a computer-implemented method and system for physicians, users/patients, and third parties to share health-related information. Some embodiments of the apparatus may be described with reference to FIGS. 1 through 4.

As shown in FIG. 1, the method and system (collectively the “system”) 100 involves interactions between a user 110 and a healthcare provider 120 or user 110 and a third party 180. System 100 includes a first device 130 belonging to healthcare provider 120, an electronic data server 140, a healthcare data specialist 150, a second device 160 belonging to user 110, and a third device 170 belonging to third party 180.

### Medical Test

In one aspect, system 100 allows healthcare provider 120 to share medical-test result with user 110 on device 160. User 110, first, must receive a medical test from healthcare provider 120. This medical test can include but is not limited to a sexually-transmitted disease test, a blood lipid level test, a pregnancy test, biopsy results, or any other medical test performed by a healthcare provider.

Healthcare provider 120 then enters the results of the medical test into device 130. Healthcare provider 120 can enter the medical-test result into device 130 using several different methods including but not limited entering the medical-test result into an app if device 130 is a mobile device, entering the medical-test result into locally run software if device 130 is a computer, or entering the medical-test result into a web-based portal. Healthcare provider 120 can only access the app, software, or web portal on device 130 after completing a verification process that proves healthcare provider's 120 legitimacy.

### Electronic Data Server and Processing of Data

After healthcare provider 120 has entered the medical-test result into device 130. Device 130 transmits the data associated with the medical-test result to server 140. Server 140 then stores the data in a database associated with server 140. Server 140 can either be a physical server, a virtual server, or a networked hybrid of a physical server and a virtual server. The data associated with the medical-test result is stored in the database using a unique identifier associated only with user 110.

Server 140 also displays the data to data specialist 150. Data specialist 150 then examines the data to verify whether or not the data reflects a positive health outcome for user 110. After examining the data, data specialist 150 will create a verified result based off of that data. The verified result will not include all of the data associated with the medical-test result, but will instead include a binary indicator as to whether or not the medical-test result reflected a positive health outcome for user 110. For example, if user 110 received a sexually-transmitted disease test from healthcare provider 120, then data specialist 150 would create a positive verified results if user 110 did not have a sexually-transmitted disease or a negative verified results if user 110 did have a sexually-transmitted disease. In one embodiment, a positive verified result is represented as a checkmark and a negative verified result is represented by the absence of a checkmark. The verified result is also stored in the database using the unique identifier associated with user 110.

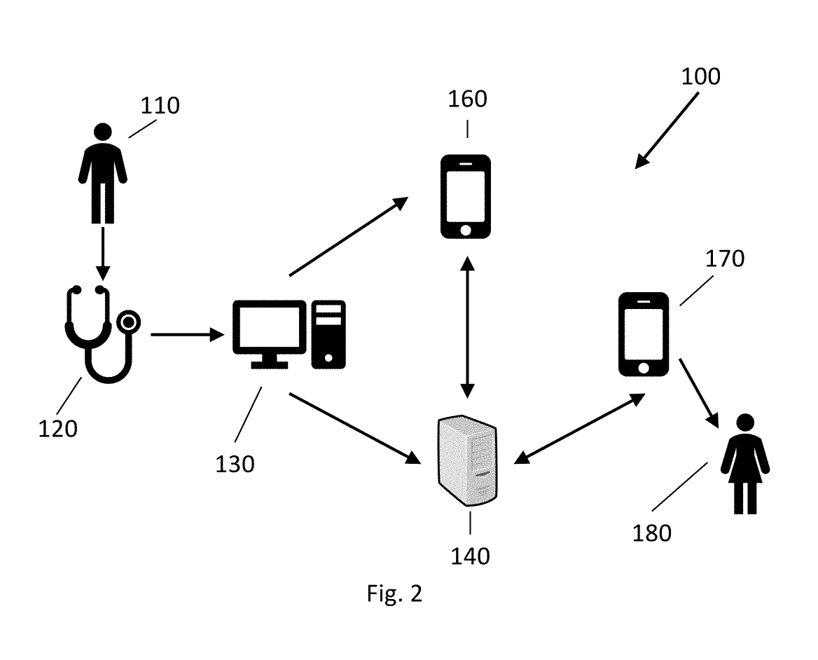

In another embodiment, shown in FIG. 2, the server processes the data associated with the medical-test result to create a verified result. The server can use several methods to create a verified result. In one, the server uses natural-language-processing tools to extract information from the medical-test result. The server then determines whether the test results reflect a positive or negative health outcome using a classifier algorithm. In another method, the server simply verifies that user 110 received a given medical test and relies on healthcare provider 120 to enter whether the result reflected a positive or negative health outcome.

In addition to storing and processing medical-test data, server 140 can perform trend analysis on the data stored in its database. For example, server 140 can look to see if there has been an uptick or downtick of the prevalence of a particular disease in a given geographic area by looking at the medical-test result uploaded from that geographic area. Server 140 can then distribute that analysis to users so that those users can make more informed healthcare decisions.

### User's Device and Sharing of Results

Returning to FIG. 1, when device 130 transmits the data associated with the medical-test result to server 140, it also transmits that data to device 160. User 110 can then access the medical-test result on device 160 using either a mobile app installed on device 160 or by logging into a web portal with device 160. After server 140 has processed the data and produced a verified result, server 140 transmits the verified result to device 160. User 110 can, once again, access the verified result through either an app or a web portal on device 160.

Through device 160, user 110 can choose to select the option to share the data or verified results with third party 180. When user 110 selects this option, server 140 transmits the data or verified result to device 170. Device 170 then displays the data or verified result to third party 180. In one embodiment, device 170 displays a positive verified result as a checkmark and a negative verified result as the absence of a checkmark. In another aspect, the color of the checkmark can represent how long ago user 110 received the medical test associated with the verified result. For example, if user 110 received the medical test less than three months ago, then the checkmark is green, if user 110 received the test three to six months ago then the checkmark is yellow and if user 110 received the test more than six months ago, then the checkmark is red. One skilled in the art would recognize that there are numerous other ways of displaying this information on device 170.

Device 160 also allows user 110 to control third party's 180 access to the medical-test data and verified results. On device 160, through either an app or web portal, user 110 can select whether the data and results are shared in a ‘limited’ or ‘unlimited’ mode. When user 110 selects ‘unlimited’ mode, all of the data and results are shared with third party 180. This includes any sensitive personal information embedded in the data and results. Third party 180 also has access to the data and results for an unlimited time period. When user 110 selects ‘limited’ mode, server 140 transmits a limited amount of information to device 170. For example, server 140 may only send the verified result and the unique identifier associated with user 110. Thereby allowing third party 180 to see the verified result without seeing user 110′s name. In another example, server 140 may send the data associated with the medical-test result with all personally identifiable information removed. The personally identifiable information may be replaced with general demographic information such as the race and age of user 110. In another aspect, device 170 may only display the data or verified result to third party 180 for a limited time, an hour for example, when user 110 selects ‘limited’ mode.

### Direct Connection Between User and Server

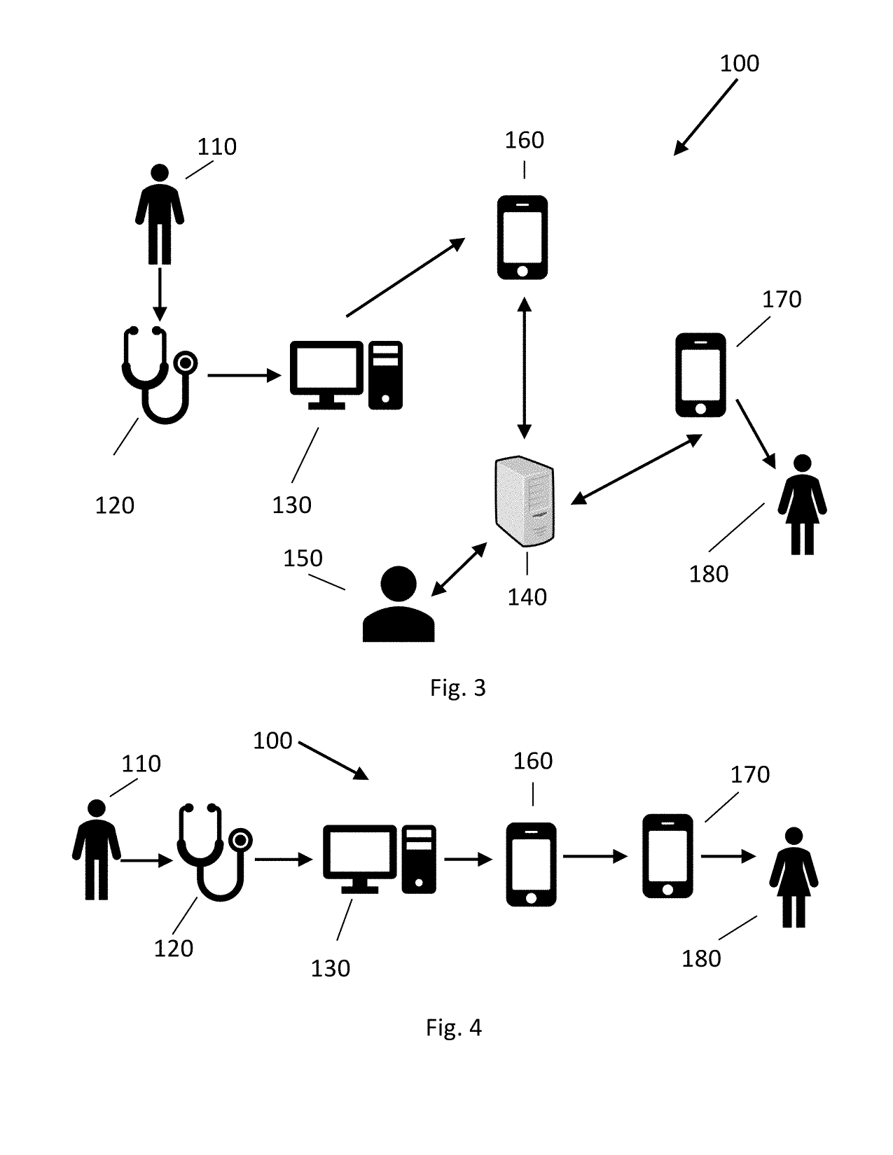

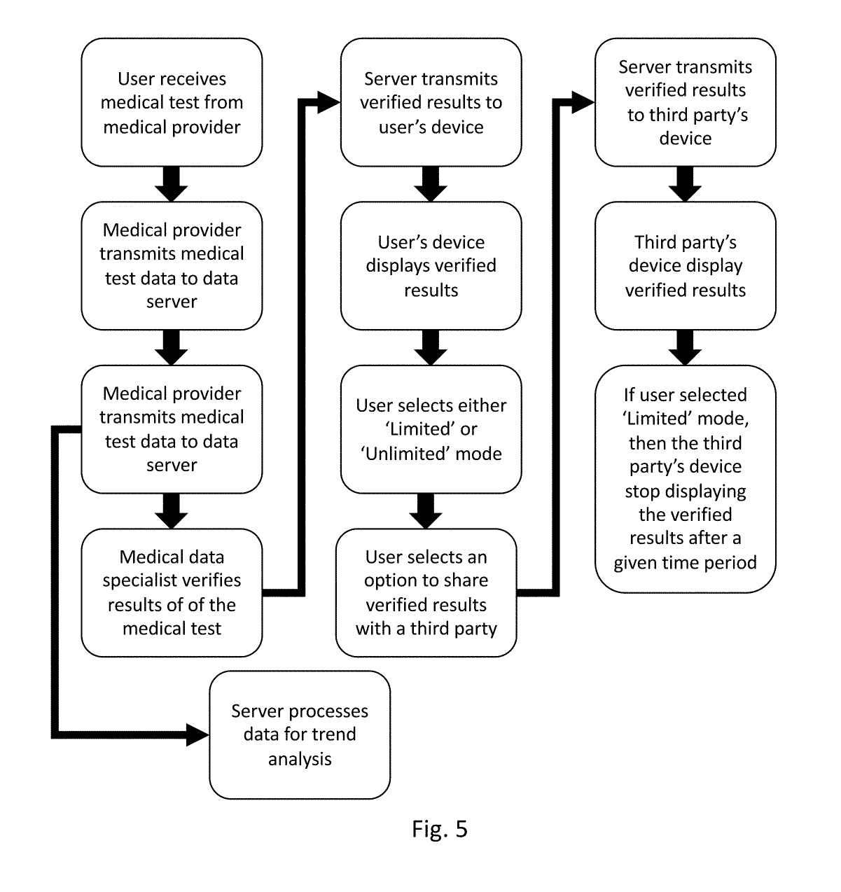

In another embodiment shown in FIG. 3, healthcare provider 120 gives user 110 a copy of the medical-test result either in paper or electronic form, but does not transmit a copy to server 140. If in electronic form, healthcare provider 120 can transmit the medical-test result from device 130 to device 160. If in paper form, user 110 can load the medical-test result into device 160 by either scanning or taking a picture of the medical-test result.

User 110 then transmits the medical-test result from device 160 to server 140. Once on server 140, data specialist 150 verifies the authenticity of the medical-test result. In one aspect, data specialist 150 or server 140 compares the medical-test result against other examples of test results from healthcare provider 120. If data specialist 150 does not have other examples from healthcare provider 120, then data specialist may contact healthcare provider 120 to obtain examples in order to verify the authenticity of the medical-test result. In another aspect, server 140 processes the medical-test result in order to search for digital signatures indicating whether or not user 110 has tampered with the medical-test result. In addition, server 140 may use natural language processing and machine learning to compare the medical-test result against previously authenticated examples.

### Direct Connection Between User and Third Party

In another embodiment shown in FIG. 4, server 140 and data specialist 150 have been removed from system 100. Instead, device 130 only communicates with device 160 and device 160 communicates directly with device 170. In this embodiment, the data associated with the medical-test result is stored on device 160 instead of server 140. In addition, healthcare provider 120 can use device 130 to create a verified result that is then transmitted to device 160 along with the data associated with the medical-test result. Device 160 then transmits the verified result or the data associated with the medical-test result depending upon whether user 110 has selected ‘limited’ or ‘unlimited’ mode. Each of the above transmissions can be done through several means such as connecting over wi-fi or near-field communication.

### Impact of System

System 100 makes it easier for user 110 to share their healthcare related information with third party 180, because all of the data is transmitted using an online system instead of with paper or over the phone. This is especially important is user 110 and third party 180 are entering into a relationship, because it allows third party 180 to know the medical status of user 110, such as whether user 110 has recently been tested for a sexually-transmitted disease (STD).

In addition, third party 180 can trust the validity of the information displayed on device 170, because healthcare provider 120 can only access the app, software, or web portal on device 130 after completing a verification process that proves healthcare provider's 120 legitimacy. This means that the test results may not be manipulated, and arrive directly from a verified healthcare provider. In other words, system 100 helps to foster authenticity, because results cannot be counterfeited or faked.

In addition, system 100 makes it easier for family members to know the medical status of user 110 if user 110 has shared that information through system 100. This is especially important if user 110 is elderly and has chronic illnesses. User 110 won't have to keep calling healthcare provider 120 (often waiting on hold) for exam or test results. Additionally, user 110 can opt out of a follow-up visit after receiving test results on device 130 if the results reflect a positive health outcome.

In yet another aspect, system 100 requests some form of verified identification (federal or state) from user 110 in order to use system 100. And user 110 has a unique identifier associated with them, which allows user 110 to more anonymously share data and results.

Finally, system 100 allows user 110 to make more informed healthcare decisions. Server 140 can provide user 110 with analysis of healthcare trends in their area. In addition, user 110, healthcare provider 120, and third party 180 can communicate with each other using system 100 further adding to user's 100 healthcare knowledge.

Those skilled in the relevant art should appreciate that they can readily use any of these disclosed embodiments as a basis for designing or modifying other structures or functions for carrying out the invention, without departing from the spirit and scope of the invention.

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

What is claimed is:

1. A computer-implemented method, comprising:
receiving, by a user, a medical test from a verified healthcare provider;
transmitting the data associated with the medical test result from a verified healthcare provider to an electronic data server over an internet connection;
processing, either by a medical data specialist or a computer processor on the server, the data to create a verified result;
transmitting the data and verified result from the server to the user's electronic device over an internet connection;
displaying the data and verified result on the user's electronic device;
selecting, by the user, one of at least a limited or unlimited mode to share the data or verified result with a third party;
selecting, by a user, an option to share the data or verified result with the third party;
transmitting the data or verified result from the server to the third party's electronic device over an internet connection; and
displaying the data or verified result on the third party's electronic device.

2. The method of claim 1, further comprising the periodic reminding of the user to return to the healthcare provider to receive another medical test.

3. The method of claim 1, where when the user selects the limited mode, the verified result is displayed to the third party for a limited time period.

4. The method of claim 1, where when the user selects the limited mode, the server removes all individually identifiable information from the verified result before transmitting it to the third party's device.

5. The method of claim 1, where the verified result is represented to the user and third party as a color-coded checkmark.

6. A computer-implemented method, comprising:
receiving, by a user, a medical test from a verified healthcare provider;
providing the user with a copy of the medical test result;
uploading the medical test result, by the user, to the user's electronic device;
transmitting the medical test result from the user's electronic device to an electronic data server over an internet connection;
authenticating, either by a medical data specialist or a computer processor on the server, the medical test result;
processing, either by a medical data specialist or a computer processor on the server, the data associated with the medical test result to create a verified result;
transmitting the data and verified result from the server to the user's electronic device over an internet connection;
displaying the data and verified result on the user's electronic device;
selecting, by the user, one of at least a limited or unlimited mode to share the data or verified result with a third party;
selecting, by a user, an option to share the data or verified result with the third party;
transmitting the data or verified result from the server to the third party's electronic device over an internet connection; and
displaying the data or verified result on the third party's electronic device.

6. The method of claim 6, further comprising the periodic reminding of the user to return to the healthcare provider to receive another medical test.

7. The method of claim 6, where when the user selects the limited mode, the data is displayed to the third party for a limited time period.

8. The method of claim 6, where when the user selects the limited mode, the data is anonymized when displayed to the third party beyond that if the user selected the unlimited mode.

9. The method of claim 6, where the verified result is represented to the user and third party as a color-coded checkmark.

10. A computer-implemented method, comprising:
receiving, by a user, a medical test from a verified healthcare provider;
transmitting data associated with a medical test result from a verified healthcare provider to a user's electronic device;
selecting, by a user, one of at least a limited or unlimited mode to share the data associated with the test result with a third party;
selecting, by a user, an option to share the data with the third party; and
displaying the data to the third party.

11. The method of claim 10, further comprising the periodic reminding of the user to return to the healthcare provider to receive another medical test.

12. The method of claim 10, where when the user selects the limited mode, the data is displayed to the third party for a limited time period.

13. The method of claim 10, where when the user selects the limited mode, the data is anonymized when displayed to the third party beyond that if the user selected the unlimited mode.
